(19) United States
(12) Patent Application Publication
(10) Pub. No.: US 2021/0147532 A1
(43) Pub. Date: May 20, 2021

(54) METHOD FOR CONTROLLING THE AFUCOSYLATION LEVEL OF A GLYCOPROTEIN COMPOSITION

(71) Applicant: ARES TRADING S.A., AUBONNE (CH)

(72) Inventors: MARTIN JORDAN, ECUBLENS (CH); HERVÉ BROLY, CHÂTEL-ST DENIS (CH); MATTHIEU STETTLER, SIERRE (CH); ELODIE CHARBAUT TALAND, VUFFLENS LE CHATEAU (CH)

(21) Appl. No.: 17/057,223

(22) PCT Filed: May 23, 2019
(86) PCT No.: PCT/EP2019/063395

§ 371 Date: Nov. 20, 2020

(30) Foreign Application Priority Data
May 24, 2018 (EP) 18173972.3

(51) Int. Cl. C07K 16/24; C12P 21/00

(52) U.S. Cl. CPC C07K 16/241; C07K 2317/41; C07K 2317/14; C12P 21/005

(57) ABSTRACT

The present invention relates to a method for controlling the afucosylation level of a glycoprotein composition. The method comprises the control of the afucosylation level by selecting the appropriate temperature and/or pH. The invention also relates to glycoprotein compositions produced according to the method of the invention.

### FIELD OF THE INVENTION

The present invention relates to a method for modulating the proportion of afucosylated species in a glycoprotein composition and to compositions obtained according to the method of the invention.

### BACKGROUND

Proteins typically undergo post-translational modifciations during their expression, including the attachment of sugar moieties. Such glycosylation can have profound effects on the biological activity of the proteins. For instance, antibody-dependent cellular cytotoxicity (ADCC), which is an important mechanism of action of many therapeutic antibodies, is dependent on the level of fucosylation of the antibody.

In particular, it has been found that monoclonal antibodies having a reduced amount of fucosylation exhibit higher ADCC as compared to their fucosylated counterparts.

There is a need to control post-translational modifications in a glycoprotein composition, such as the level of afucosylation.

### SUMMARY OF THE INVENTION

The present inventors have found that changing the temperature and/or pH allows the controlled modulation of the level of protein afucosylation.

In particular, the present disclosure relates to the following:

1. A method for controlling the afucosylation level of a glycoprotein composition according to (A) or (B):

(A) a method for increasing the afucosylation level of a glycoprotein composition compared to a reference afucosylation level of the glycoprotein composition, wherein the method comprises culturing eukaryotic cells expressing the glycoprotein at a temperature and/or pH that is lower than the pH and/or temperature used for culturing said cells expressing the glycoprotein whose afucosylation level is the reference afucosylation level; or

(B) a method for decreasing the afucosylation level of a glycoprotein composition compared to a reference afucosylation level of the glycoprotein composition,

wherein the method comprises culturing eukaryotic cells expressing the glycoprotein at a temperature and/or pH that is higher than the pH and/or temperature used for culturing said cells expressing the glycoprotein whose afucosylation level is the reference afucosylation level.

2. The method according to item 1, wherein only the temperature is (A) lower or (B) higher than the temperature used for culturing said cells expressing the glycoprotein whose afucosylation level is the reference afucosylation level and the pH is the same.
3. The method according to item 1, wherein only the pH is (A) lower or (B) higher than the pH used for culturing said cells expressing the glycoprotein whose afucosylation level is the reference afucosylation level and the temperature is the same.
4. The method according to item 1, werein both the temperature and pH are (A) lower or (B) higher than the pH and temperature used for culturing said cells expressing the glycoprotein whose afucosylation level is the reference afucosylation level.
5. The method according to any one of items 1 to 4, werein the eukaryotic cells are mammalian cells.
6. The method according to item 5, werein the mammalian cells are CHO cells.
7. The method according to any one of items 1 to 6, werein the glycoprotein is an antibody or antibody fragment.
8. The method according to any one of items 1 to 7, werein the change in pH and/or temperature is limited to the production phase.
9. A glycoprotein composition obtainable by a method according to any one of items 1 to 8.
10. A kit comprising the glycoprotein composition according to item 9 and instructions for use.

Any features, including optional, suitable, and preferred features, described in relation to any particular aspect of the invention may also be features, including optional, suitable and preferred features, of any other aspect of the present invention.

### BRIEF DESCRIPTION OF THE FIGURES

FIG. 1 shows the combined effects of pH and temperature on the level of total afucosylated glycans (=A0+A1+A2+M4+M5+M6+M7+M8). The indicated pH refers to the upper pH limit applied between day 5 and day 17 of culture.

FIG. 2 shows the combined effects of pH and temperature on the level of total high mannose glycans (=M4+M5+M6+M7+M8).

FIG. 3 shows the combined effects of pH and temperature on the level of M6 glycoform (high mannose species containing 6 mannose sugars).

FIG. 4 shows the level of total afucosylated glycans (=A0+A1+A2+M4+M5+M6+M7+M8) and the level of total high mannose glycans (=M4+M5+M6+M7+M8) of adalimumab samples from the low afucosylation process and the high afucosylation process.

FIG. 5 shows the level of total galactosylated glycans (=“FA2G1-1”+“FA2G1-2”+FA2G2+“Hybrid-F”) and the total level of G0 of adalimumab samples from the low afucosylation process and the high afucosylation process.

FIG. 6 shows the distribution of charge variants of adalimumab samples from the low afucosylation process and the high afucosylation process.

FIG. 7 shows the level of afucosylated glycans of adalimumab samples from the low afucosylation process and the high afucosylation process.

### DETAILED DESCRIPTION OF THE INVENTION

The present invention, in some embodiments thereof, relates to a method for controlling the level of afucosylation of a glycoprotein composition, as well as glycoprotein compositions obtained according to such method.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

In order to control the level of afucosylation of a glycoprotein composition, the present inventors investigated into manipulating the glycoprotein production process in various ways, including the addition of media feeds and modulation of parameters during the cell culture process. Most tested parameters had no impact on the level of afucosylation. However, the present inventors observed a correlation between the level of afucosylation and the pH or temperature of the cell culture expressing the glycoprotein. In particular, it was found that the manipulation of either parameter correlates with a change in the level of afucosylation and that the modification of both parameters in combination results in an even increased change in the level of afucosylation as compared to a manipulation of either parameter alone. Without being bound by theory, it is believed that the effect of the temperature and the pH on the afucosylation level at least partially relies on the stress that is put on the Golgi apparatus and is thus independent of the particular glycoprotein that is expressed or the particular cell that is used for expressing the glycoprotein.

Thus, the present invention relates to a method for controlling the level of afucosylation of a glycoprotein composition by modifying the temperature and/or pH of the cell culture expressing the glycoprotein. Preferably, both the pH and temperature are modified.

Provided herein is a method for increasing the level of afucosylation of a glycoprotein composition by decreasing the pH and/or temperature of the cell culture expressing the glycoprotein. Also provided herein is a method for decreasing the level of afucosylation of a glycoprotein composition by increasing the pH and/or temperature of the cell culture expressing the glycoprotein. In some embodiments, the pH and/or temperature is decreased or increased as compared to the pH and/or temperature of a cell culture expressing the glycoprotein and whose level of afucosylation serves as a reference value.

Provided is a method for controlling the level of afucosylation of a glycoprotein composition comprising culturing cells expressing said glycoprotein and adjusting the temperature and/or pH of the cell culture to match a desired afucosylation level of the glycoprotein composition.

Provided is a method for controlling the level of afucosylation of a glycoprotein composition comprising the following steps:
(a) comparing the level of afucosylation of a glycoprotein composition obtained by culturing cells expressing said glycoprotein at an initial temperature and/or pH to a desired afucosylation level;
(b) determining if the level of afucosylation obtained by culturing cells expressing said glycoprotein at an initial temperature and/or pH is below or above the desired afucosylation level; and,
(c) (i) if the level of afucosylation obtained by culturing cells expressing said glycoprotein at an initial temperature and/or pH is below the desired afucosylation level, culturing cells expressing said glycoprotein at a temperature and/or pH that is lower than the initial temperature and/or pH; or
   (ii) if the level of afucosylation obtained by culturing cells expressing said glycoprotein at an initial temperature and/or pH is above the desired afucosylation level, culturing cells expressing said glycoprotein at a termperature and/or pH that is higher than the initial temperature and/or pH.

Generally known criteria, such as the viability of the cells and the protein yield, are considered for selecting the temperature and pH for culturing the cells in the methods of the invention. The culture process is typically divided into a growth and a production phase. During the growth phase, conditions are selected that promote exponential growth of the cells, whereas during the production phase conditions are selected that promote protein production.

Apart from generally known criteria for selecting the temperature and pH, the desired afucosylation level of the glycoprotein composition is further considered for selecting the temperature and/or pH of the culture process of the invention. As outlined above, a lower pH and/or temperature can be selected if a higher level of afucosylation is desired and a higher pH and/or temperature can be selected if a lower level of afucosylation is desired.

The adjustment of the temperature and/or pH in accordance with the desired afucosylation level may extend to the whole culture process or be limited to a part of the process, e.g., to the production phase. The pH and/or temperature may also be adjusted more than once. For instance, after an initial temperature and pH during the growth phase of the cells, the pH and/or temperature may be adjusted to a certain value at the beginning of the production phase and then adjusted to another value later during the production phase. The pH and/or temperature adjustment may also occur passively and/or gradually, such as the natural pH drop that is observed during cell growth.

The pH and/or temperature chosen in the methods of the invention may be identical with the pH and/or temperature that is conventionally used for culturing cells.

Also provided is a method of producing a glycoprotein, wherein the cell producing the glycoprotein is cultured at a low temperature and/or low pH. The glycoprotein compositions produced according to this method have a particularly high afucosylation level.

In one embodiment, the temperature is in the range of 28-34° C. during at least part of the culture process, e.g., during at least part of the production phase. Preferably, the temperature is in the range of 28-30° C., more preferably about 29° C., during at least part of the production phase. In further embodiments, the temperature during the production phase is first in the range of 32-34° C., preferably about 33° C., for at least one day and then in the range of 28-30° C., preferably about 29° C., for at least another day.

In one embodiment, the pH is in the range of pH 6.6-6.9 during at least part of the culture process, e.g., during at least part of the production phase. Preferably, the pH is in the range of pH 6.65-6.8, more preferably about pH 6.7-6.75, during at least part of the production phase, preferably the entire production phase.

In one embodiment, the temperature is in the range of 28-34° C. and the pH is in the range of pH 6.6-6.9 during at least part of the production phase. Preferably, the temperature is in the range of 28-30° C., more preferably about 29° C., and the pH is in the range of pH 6.65-6.8, more preferably about pH 6.7-6.75, during at least part of the production phase, preferably the entire production phase. In some embodiments, the temperature during the production phase is first in the range of 32-34° C., preferably about 33° C., for at least one day and then in the range of 28-30° C., preferably about 29° C., for at least another day and the pH is about pH 6.7-6.75 during the entire production phase.

The cell that is used in a method of the invention is a eukaryotic cell, preferably one having a Golgi apparatus. Preferably, the cell is a mammalian cell, in particular, a mammalian cell line. Exemplary cell lines include CHO, HeLa, COS, NS0, SP0, NIH 3T3, HT1080, A549, U2OS, HEK293, P19, CAD, J558L, N2a, SO-Rb50, Y79, Hep G2, PER.C6, HKB-11, CAP, HuH-7 and L929. Most preferably, the used cell line is a CHO cell line.

A Chinese hamster ovary tissue-derived CHO cell or cell line suitable in accordance with the present invention is any cell which is a cell line established from an ovary tissue of Chinese hamster (Cricetulus griseus). Examples include CHO cells described in documents such as Journal of Experimental Medicine, 108, 945 (1958); Proc. Nat Acad. Sci. USA, 60, 1275 (1968); Genetics, 55, 513 (1968); Chromosoma, 41, 129 (1973); Methods in Cell Science, 18, 115 (1996); Radiation Research, 148, 260 (1997); Proc. Nat Acad. Sci. USA, 77, 4216 (1980); Proc. Nat Acad. Sci., 60, 1275 (1968); Cell, 6, 121 (1975); Molecular Cell Genetics, Appendix I, II (pp. 883-900); and the like. In addition, CHO-K1 (ATCC CCL-61), DUXB1 1 (ATCC CCL-9096) and Pro-5 (ATCC CCL-1781) registered in ATCC (The American Type Culture Collection) as well as CHO-S (Life Technologies, Cat #1 1619) or sub-cell lines obtained by adapting the cell lines using various media can also be employed in the present invention.

In some embodiments, the host cell is a CHO-1E5, CHO-S, CHO/DG44, CHO-3F, or CHO-2.6 clone.

Following expression of the glycoprotein under the modified cell culture conditions of the invention, the cells expressing the glycorprotein may be harvested and the glycoprotein purified according to conventional means.

“Glycoprotein” refers to a protein that is modified with a sugar moiety. In some embodiments, the glycoprotein has therapeutic use. In some embodiments, the glycoprotein is selected from the group consisting of an antibody, antibody fragment, enzyme, receptor, hormone, regulatory factor and growth factor. Preferably, the glycoprotein is an antibody.

“Antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact polyclonal or monoclonal antibodies, but also, unless otherwise specified, any antigen-binding fragment or antibody fragment thereof that competes with the intact antibody for specific binding, fusion proteins comprising an antigen-binding portion (e.g., antibody-drug conjugates), any other modified configuration of the immunoglobulin molecule that comprises an antigen recognition site, antibody compositions with poly-epitopic specificity, and multi-specific antibodies (e.g., bispecific antibodies).

“Antigen-binding fragment” of an antibody or “antibody fragment” comprises a portion of an intact antibody, which is still capable of antigen binding and/or the variable region of the intact antibody. Antigen-binding fragments include, for example, Fab, Fab′, F(ab′)2, Fd, and Fv fragments, domain antibodies (dAbs, e.g., shark and camelid antibodies), fragments including complementarity determining regions (CDRs), single chain variable fragment antibodies (scFv), single-chain antibody molecules, multi-specific antibodies formed from antibody fragments, maxibodies, minibodies, intrabodies, diabodies, triabodies, tetrabodies, v-NAR and bis-scFv, linear antibodies (see e.g., U.S. Pat. No. 5,641,870, Example 2; Zapata et al. (1995) Protein Eng. 8HO: 1057), and polypeptides that contain at least a portion of an immunoglobulin that is sufficient to confer specific antigen binding to the polypeptide. Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site. Pepsin treatment of an antibody yields a single large F(ab′)2 fragment, which roughly corresponds to two disulfide linked Fab fragments having different antigen-binding activity and is still capable of cross-linking antigen. Fab′ fragments differ from Fab fragments by having a few additional residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments were originally produced as pairs of Fab′ fragments which have hinge cysteines between them. Other chemical couplings of antibody fragments are also known.

Humanized forms of non-human (e.g., murine) antibodies are chimeric molecules of immunoglobulins, immunoglobulin chains or fragments which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

In some embodiments, the antibody is an inhibitory antibody. Inhibitory antibody may inhibit one or more biological activities of the antigen to which the antibody binds. For example, an inhibitory antibody can downregulate signal transduction of the corresponding antigen by inhibiting the activity of the antigen or inhibit expression of the antigen. In some embodiments, the antibody is a neutralizing antibody. A neutralizing antibody reduces or abolishes some biological activity of a soluble antigen or of a living microorganism, such as an infectious agent. Neutralizing antibodies may compete with the natural ligand or receptor for its antigen. In some embodiments, the antibody is a stimulatory or activating antibody. A stimulatory or activating antibody may be an agonist antibody which may activate signal transduction of the corresponding antigen upon binding of the antigen thereby activating or upregulating the activity of the antigen, or upregulate the expression of the antigen to which the antibody binds.

In one embodiment, the light and heavy chains may be transformed into separate modified host cell cultures, either of the same or of differing species. In an alternative embodiment, separate plasmids for light and heavy chain may be used to co-transform a single modified host cell culture. In another embodiment, a single expression plasmid containing both genes and capable of expressing the genes for both light and heavy chain may be transformed into a single modified host cell culture.

When heavy and light chains are coexpressed in the same host, the isolation procedure is designed so as to recover reconstituted antibody. This can be accomplished by conventional antibody purification procedures such as, for example, protein A-Sepharose, hydroxylapatite chromatography, gel electrophoresis, dialysis, or affinity chromatography.

The antibody may bind an antigen such as a cancer antigen. The cancer antigen may be selected from the group consisting of PD-1, PD-L1, HER2, Immunoglobulin epsilon Fc receptor II, Alk-1, CD20, EGF receptor, VEGF receptor, FGF receptor, NGF receptor, PDGF receptor, EpCam, CD3, CD4, CD 11 a, CD 19, CD22, CD30, CD33, CD38, CD40, CD51, CD55, CD80, CD95, CCR2, CCR3, CCR4, CCR5, CTLA-4, Mucin 1, Mucin 16, Endoglin, Mesofhelin receptor, Nogo receptor, folate receptor, CXCR4, insulin-like growth factor receptor, Ganglioside GD3, and alpha and beta integrins.

Exemplary antibodies produced in the cells of the present invention include, but are not limited to, alemtuzumab, atezolizumab, avelumab, basiliximab, cemiplimab, cetuximab, daclizumab, dacetuzumab, durvalumab, efalizumab, epratuzumab, ibritumomab, tiuxetan, infliximab; muromonab-CD3 (OKT3), nivolumab, omalizumab, palivizumab, pembrolizumab, oregovomab, rituximab, trastuzumab, ocrelizumab, pertuzumab, hu M195Mab, anti-Abeta, anti-CD4, anti-oxLDL, trastuzumab-DMI, apomab, rhuMAb GAIOI, anti-OX40L, ipilimumab, ofatumumab, zalutumumab, motavizumab, ecromeximab, MDXOIO, 4B5, TNX-901, and IDEC-114.

The term “fucosylation level” refers to the proportion of glycans in a protein composition which carry a fucose modification. Likewise, “afucoslyation level” refers to the proportion of glycans in a protein composition without a fucose modification. In some embodiments, the proportion of glycans without a fucose modification may be calculated as the sum of A0, A1, A2, M4, M5, M6, M7 and M8 glycans divided by the total glycans.

The present invention also provides glycoprotein compositions that are obtained according to a method of the invention.

The glycoprotein composition may comprise a pharmaceutically-acceptable carrier. “Pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof.

The compositions of the present disclosure may be in a variety of forms. These include, for example, liquid, semi-solid and solid dosage forms, such as liquid solutions (e.g., injectable and infusible solutions), dispersions or suspensions, tablets, pills, powders, liposomes, and suppositories. The preferred form depends on the intended mode of administration and therapeutic application. Typical preferred compositions are in the form of injectable or infusible solutions, such as compositions similar to those used for passive immunization of humans. The preferred mode of administration is parenteral (e.g., intravenous, subcutaneous, intraperitoneal, or intramuscular). In a preferred embodiment, the composition is administered by intravenous infusion or injection. In another preferred embodiment, the composition is administered by intramuscular or subcutaneous injection.

Liquid dosage forms for oral administration include, but are not limited to, pharmaceutically acceptable emulsions, microemulsions, solutions, suspensions, syrups and elixirs. In addition to the glycoprotein, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, lavouring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of the glycoprotein, it is often desirable to slow absorption from subcutaneous or intramuscular injection. This may be accomplished by the use of a liquid suspension of crystalline or amorphous material with poor water solubility. The rate of absorption then depends upon its rate of dissolution that, in turn, may depend upon crystal size and crystalline form. Alternatively, delayed absorption of parenterally administered glycoprotein is accomplished by dissolving or suspending the compound in an oil vehicle. Injectable depot forms are made by forming microencapsule matrices of glycoprotein in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories, which can be prepared by mixing the compounds of this invention with suitable non-irritating excipients or carriers such as cocoa butter, polyethylene glycol or a suppository wax, which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol and silicic acid, b) binders such as, for example, carboxymethylcellulose, alginates, gelatin, polyvinylpyrrolidinone, sucrose and acacia, c) humectants such as glycerol, d) disintegrating agents such as agar-agar, calcium carbonate, potato or tapioca starch, alginic acid, certain silicates and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hardfilled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The glycoprotein can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms, the glycoprotein may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of the glycoprotein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Typically, the glycoprotein is incorporated into pharmaceutical compositions suitable for administration to a subject, wherein the pharmaceutical composition comprises the glycoprotein and a pharmaceutically acceptable carrier. In many cases, it is preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the glycoprotein.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the glycoprotein in the required amount in an appropriate solvent with one or a combination of ingredients enumerated above, as required, followed by filtered sterilization. Generally, dispersions are prepared by incorporating the active ingredient into a sterile vehicle that contains a basic dispersion medium and the required other ingredients from those enumerated above. In the case of sterile powders for the preparation of sterile injectable solutions, the preferred methods of preparation are vacuum drying and freeze-drying that yield a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion, and by the use of surfactants. Prolonged absorption of injectable compositions can be brought about by including in the composition an agent that delays absorption, for example, monostearate salts and gelatin.

In a further aspect, the invention relates to a kit comprising the glycoprotein composition and a package insert comprising instructions for use or for administering the glycoprotein composition.

In a further aspect, the invention relates to the use of the glycoprotein composition in a method of treatment.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

The terms “comprises”, “comprising”, “includes”, “including”, “having” and their conjugates mean “including but not limited to”.

“About” when used to modify a numerically defined parameter refers to a minor alteration of the parameter. In some embodiments, the term “about” allows the defined parameter to vary by as much as 10% preferably by as much as 5% below or above the stated numerical value for that parameter. When a parameter is defined by use of the antecedent “about”, the particular value forms another aspect.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range.

Whenever a numerical range is indicated herein, it is meant to include any cited numeral (fractional or integral) within the indicated range. The phrases “ranging/ranges between” a first indicate number and a second indicate number and “ranging ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

### Example 1

CHO cells expressing adalimumab were kept in fed-batch culture. The cells were cultured under identical conditions except for the temperature and pH, which were varied as follows.

The temperature was maintained at 37° C. from the beginning of the culture until day 5. From day 5 until day 7 of culture it was maintained at 33° C. and then it was maintained at 27, 29, 31 or 33° C. until harvest on day 17.

The pH was maintained between pH 6.9 and pH 7.2 from the beginning of the culture until day 5 of culture. From day 5 until harvest on day 17, the pH was maintained in the range of pH 6.55-6.6, pH 6.65-6.7, pH 6.75-6.8, pH 6.85-6.9 or pH 7.15-7.2.

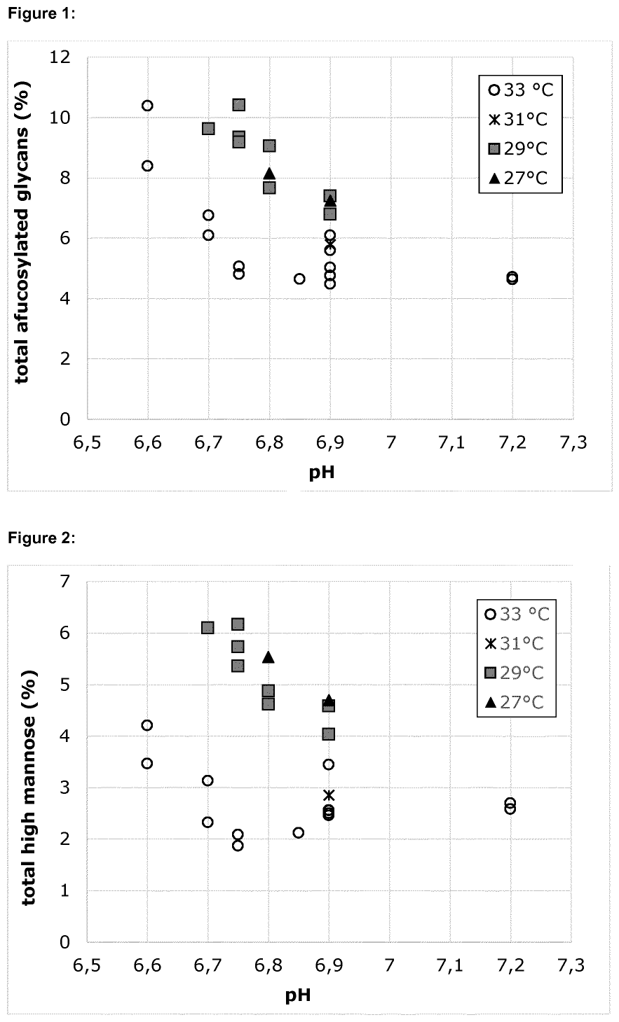

After harvest, adalimumab was purified and the glycans of each sample were quantified. The level of total afucosylation (sum of A0, A1, A2, M4, M5, M6, M7 and M8 glycans) in each sample is shown in FIG. 1. As reflected in this Figure, the afucosylation increases with decreasing pH and/or temperature.

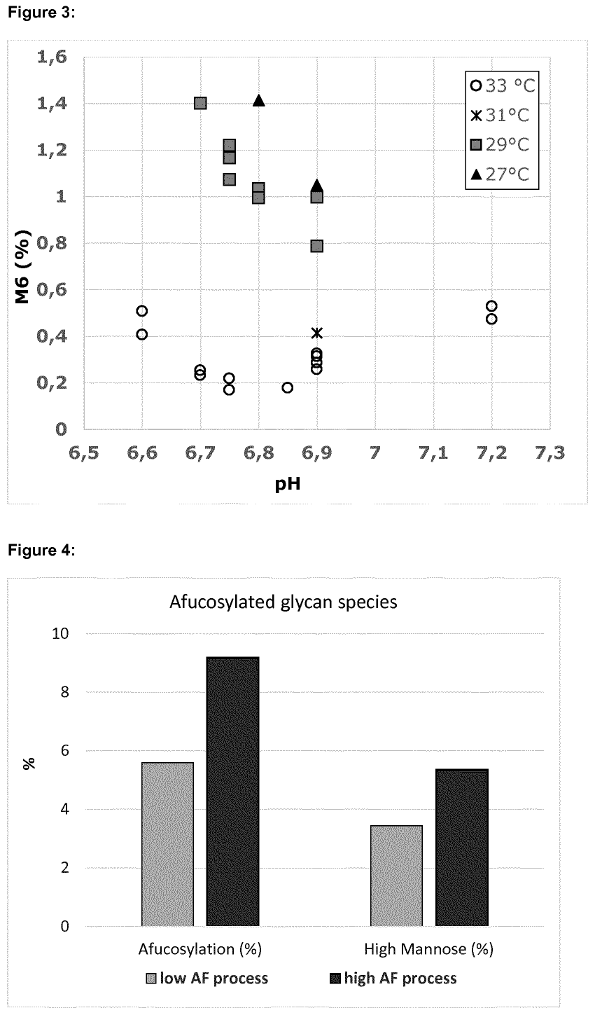

The level of total high mannose (sum of M4, M5, M6, M7 and M8 glycans) in each sample is shown in FIG. 2 and the level of the M6 glycoform is shown in FIG. 3.

### Example 2

CHO cells expressing adalimumab were kept in fed-batch culture. The cells were cultured under identical conditions except for the temperature and pH, which were varied as follows.

In a process referred to as low afucosylation (AF) process, the temperature was maintained at 37° C. from the beginning of the culture until day 5 and then maintained at 33° C. until harvest on day 17. The pH was maintained in the range of pH 6.9-7.2 from the beginning of the culture until day 5 and then maintained in the range of pH 6.85-6.9 until harvest on day 17.

In a process referred to as high afucosylation (AF) process, the temperature was maintained at 37° C. from the beginning of the culture until day 5, then maintained at 33° C. until day 7 and finally maintained at 29° C. until harvest on day 17. The pH was maintained in the range of pH 6.9-7.2 from the beginning of the culture until day 5 and then maintained in the range of pH 6.7-6.75 until harvest on day 17.

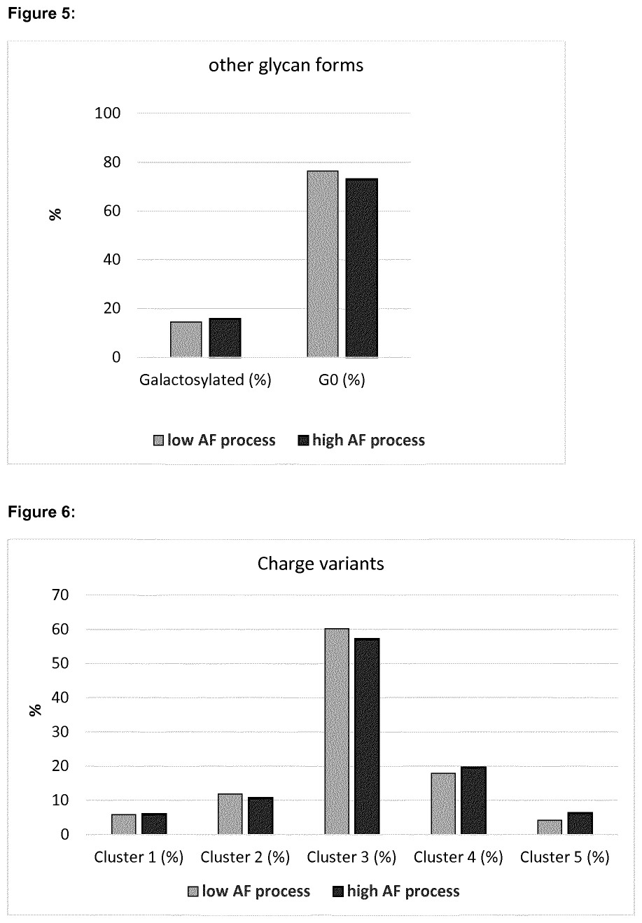

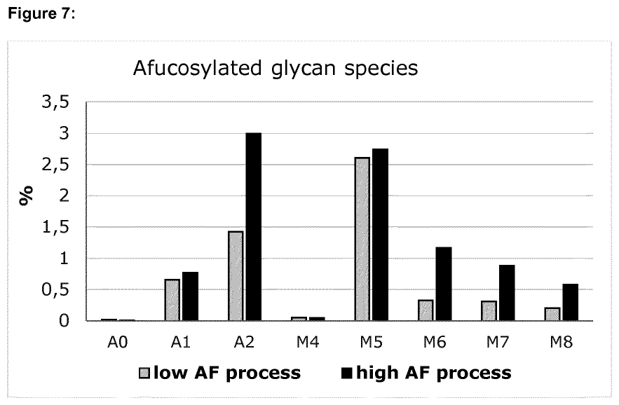

After harvest, adalimumab was purified and the glycans and charge variants of each sample were quantified. As reflected by FIGS. 4 to 7, controlling pH and temperature allows to specifically modify the level of total afucosylation without a significant effect on other quality parameters, such as the overall distribution of charge variants or the level of other glycan species, e.g., galactosylated glycans.

1-11. (canceled)

12. A method for controlling the afucosylation level of a glycoprotein composition according to (A) or (B):
(A) a method for increasing the afucosylation level of a glycoprotein composition compared to a reference afucosylation level of the glycoprotein composition,
wherein the method comprises culturing eukaryotic cells expressing the glycoprotein at a temperature and/or pH that is lower than the pH and/or temperature used for culturing said cells expressing the glycoprotein whose afucosylation level is the reference afucosylation level; or
(B) a method for decreasing the afucosylation level of a glycoprotein composition compared to a reference afucosylation level of the glycoprotein composition,
wherein the method comprises culturing eukaryotic cells expressing the glycoprotein at a temperature and/or pH that is higher than the pH and/or temperature used for culturing said cells expressing the glycoprotein whose afucosylation level is the reference afucosylation level.

13. The method according to claim 12, wherein only the temperature is changed compared to the temperature used for culturing said cells expressing the glycoprotein whose afucosylation level is the reference afucosylation level and the pH is kept the same.

14. The method according to claim 12, wherein only the pH is changed compared to the pH used for culturing said cells expressing the glycoprotein whose afucosylation level is the reference afucosylation level and the temperature is kept the same.

15. The method according to claim 12, wherein both the temperature and pH are changed as compared to the pH and temperature used for culturing said cells expressing the glycoprotein whose afucosylation level is the reference afucosylation level.

16. The method according to claim 12, wherein the eukaryotic cells are mammalian cells.

17. The method according to claim 16, wherein the mammalian cells are CHO cells.

18. The method according to claim 12, wherein the glycoprotein is an antibody or antibody fragment.

19. The method according to claim 12, wherein the change in pH and/or temperature is limited to the production phase.

20. A glycoprotein composition obtainable by a method according to claim 12.

21. A kit comprising the glycoprotein composition according to claim 20 and instructions for use.

22. A method for controlling the level of afucosylation of a glycoprotein composition comprising a change in temperature and/or pH while culturing eukaryotic cells.
